(19) United States
(12) Patent Application Publication
(10) Pub. No.: US 2026/0314994 A1
(43) Pub. Date: Oct. 15, 2026

(54) Device For Injecting Into The Skin By Superficial Incision Having Mobile Incision Components

(71) Applicant: A2M, Saint-Mesmin (FR)

(72) Inventor: Thierry Sourisseau, Saint-Mesmin (FR)

(73) Assignee: A2M, Saint-Mesmin (FR)

(21) Appl. No.: 18/876,882

(22) PCT Filed: Jun. 26, 2023
(86) PCT No.: PCT/FR2023/050962

§ 371 Date: Dec. 19, 2024

(30) Foreign Application Priority Data
Jun. 28, 2022 (FR) FR2206490

(51) Int. Cl. A61B 5/00; A61B 17/3209

(52) U.S. Cl. CPC A61B 5/411; A61B 17/32093

(57) ABSTRACT

A device (1) for injecting into the skin by superficial incision, the device (1 ) comprising an incision component (220, 230) comprising a tip (222, 232) oriented LA towards the outside of the device, the tip (222, 232) defining an ejection channel (280), the component defining a reservoir channel (282), the device (1) being configured to move the component when the tip is applied to the skin of a human or animal body, such that a volume of the ejection channel (280) is increased and a volume of the reservoir channel (282) is decreased.

### FIELD OF THE INVENTION

The present invention concerns the field of devices for superficial incision of the skin of a human or animal body, especially for diagnosing an allergy. Skin allergic diagnostic tests are generally referred to as prick tests.

### STATE OF THE ART

Allergic diagnostic tests are medical examinations consisting of the superficial penetration of a few micro-droplets of an allergenic product into the upper part of the epidermis of a human or animal body, for example in the forearm, in order to test the reactivity of the human or animal to said product.

In practice, after depositing a drop of product on the skin of a human or animal body, the skin is pricked through the drop with a sharp device, to make the product penetrate into the skin, then there is a wait of fifteen to twenty minutes before interpreting the skin reaction.

These test devices present a risk of false negative error, i.e., the risk of incorrectly reporting that an allergic individual is not allergic, when the test product does not penetrate or does not penetrate sufficiently into the skin.

Incision devices containing a vaccine solution are also available on the market, the incision device being capable of incising skin and simultaneously penetrating the vaccine into the skin.

These vaccine devices present a risk of non-vaccination when the vaccine solution does not penetrate or does not penetrate sufficiently into the skin.

For these two types of devices, the active product, i.e. the allergenic product or the vaccine solution, can be retained in the device by surface tensions between the test product and a wall of the device.

There is therefore a need to have a device for superficial incision of the skin of a human or animal body which allows sufficient penetration of an active product into the skin.

### DISCLOSURE OF THE INVENTION

One object of the invention is to propose a device for the superficial incision of the skin of a human or animal body which allows sufficient penetration of an active product into the skin.

The object of the present invention is achieved by means of a device for injecting into the skin by superficial incision, the device comprising an incision component comprising a tip oriented toward the outside of the device, the tip defining an ejection channel, the component defining a reservoir channel, the device being configured to move the component when the tip is applied to the skin of a human or animal body, such that a volume of the ejection channel is increased and a volume of the reservoir channel is decreased. The device comprises a reservoir channel and an ejection channel defined by the tip. By moving the component, the device can evolve in such a way that the volume of the ejection channel increases and the volume of the reservoir channel decreases. An active product initially in the reservoir channel can thus be transported to the ejection channel and ultimately the skin incised by the tips. The quantity of active product that penetrates the skin increases.

Such a device is advantageously and optionally supplemented by the following different characteristics taken alone or in combination:
the tip is located on a first side of a separation plane passing through the ejection channel, the incision component being mounted so as to be rotatable relative to a body of the device along a pivot zone, the pivot zone being located outside the separation plane between the ejection channel and the reservoir channel;
a lever located on the first side of the separation plane, the lever being rigidly connected to the component and the lever being connected to the body of the device by the pivot zone, the lever being configured to move the component when the lever comes into contact with the skin;
a means of locking the lever;
the locking means comprises complementary hooks on the body of the device and on an arm extending from one end of the lever;
the component has a first wall which defines the reservoir channel, the first wall having a shape complementary to a second wall of the device located opposite the first wall;
one of the first wall and the second wall has a convex zone opposite a concave zone of the other wall.

The invention also relates to a method of surface incision in which a tip of a device for injecting into the skin by surface incision is applied to the skin of a human or animal body, the application causing a reduction in a volume of a reservoir channel and an increase in a volume of an ejection channel, the ejection channel being defined by the tip, so as to transport a liquid from the reservoir channel into the ejection channel.

### DESCRIPTION OF THE FIGURES

Other characteristics and advantages of the invention will emerge from the following description, which is purely illustrative and non-limiting, and which must be read with reference to the attached drawings in which:

FIG. 1 is a schematic representation of a first cavalier view of an incision device according to a first embodiment of the invention;

FIG. 2 shows a partial enlarged-scale detail view of one end of an incision device;

FIGS. 3 and 4 show the device at two successive steps of use for a skin incision.

### DETAILED DESCRIPTION OF THE INVENTION

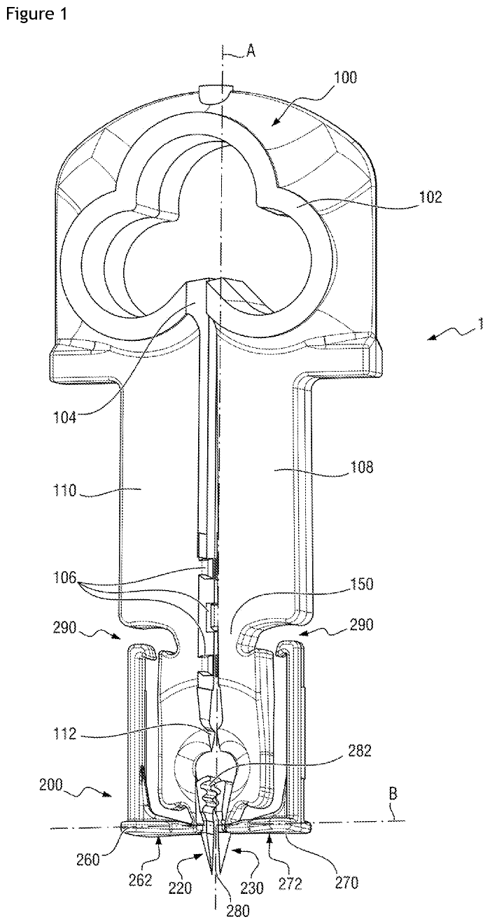

With reference to FIGS. 1 to 4, a main embodiment of the invention will be described. These figures illustrate a device 1 for injecting into the skin by superficial incision.

A longitudinal axis A is defined, defining a longitudinal direction, along which axis the surface incision device 1 extends.

### Incision Components

The device 1 comprises at least one incision component 220, 230 located at a longitudinal end of the device.

The device 1 can comprise two incision components 220, 230 as shown in FIG. 1, or more incision components.

As an option, the device can comprise a handle 100, i.e., a manual gripping zone, at the other longitudinal end of the device 1.

The incision component(s) 220, 230 form part of an incision assembly 200 of the device 1.

The device can be described as consisting of the incision assembly 200 comprising the incision component(s) 220, 230, and of a body 150 of the device which can comprise, as applicable, the handle 100.

For example, the incision component extends opposite a fixed part of the device 1 in the longitudinal direction. The component can be separated from this fixed part by a gap in a lateral axis B, defining a lateral direction. This lateral gap between the tip and the fixed part makes it possible to define an ejection channel.

Lateral axis B is orthogonal to longitudinal axis A. A third axis C, or transverse axis, can be defined, which is orthogonal to longitudinal axis A and lateral axis B.

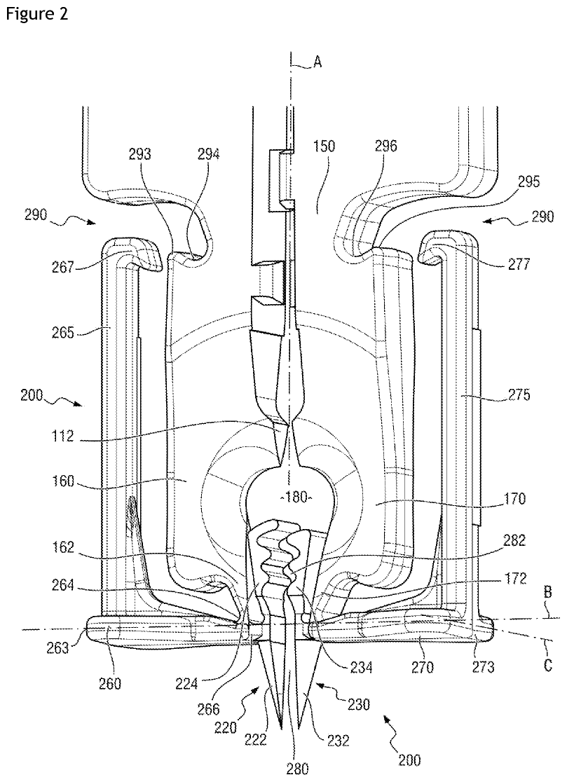

In the case where the device comprises two incision components, and as illustrated in FIGS. 1 and 2, these components extend opposite each other in the longitudinal direction. The components can be separated by a gap in a lateral axis B, defining a lateral direction.

Each component has a tip facing outwards from the device. The tips 222, 232 project from the device 1 and can be directed in the longitudinal direction.

The two tips 222, 232 are separated so as to define between them the ejection channel 280.

The component 220, 230 also defines a reservoir channel 282 which is not defined by the tip 222, 232. The reservoir channel 282 is further inside the device 1 than the ejection channel 280. In other words, the reservoir channel 282 is located between the ejection channel 280 and the body of the device 150.

For example, the component 220, 230 can comprise a press 224, 234 which is an extension of the material of the tip in the longitudinal direction. The press 224, 234 is located between the tip and the body of the device 150. The press 224, 234 extends longitudinally opposite a fixed part and is separated from the fixed part by a lateral gap along the lateral axis B. This lateral gap makes it possible to define the reservoir channel 282.

In the case where the device comprises two incision components, and as illustrated in FIGS. 1 and 2, each component 220, 230 comprises a press 224, 234. The presses 224, 234 are located between the tip and the body of the device 150. The presses 224, 234 extend longitudinally opposite each other and are separated by a lateral gap along the lateral axis B. This lateral gap makes it possible to define the reservoir channel 282.

The reservoir channel and the ejection channel are connected in such a way that a liquid in the reservoir channel can be transported into the ejection channel and vice versa. In the example of FIG. 1, in the longitudinal extension, the reservoir channel is in the longitudinal extension of the ejection channel.

The component 220, 230 is mounted to move relative to the body of the device 1. In particular, the component 220, 230 can move between a neutral position and an injection position.

By passing from the neutral position to the injection position, a volume of the ejection channel 280 is increased and a volume of the reservoir channel 282 is decreased.

For example, the passage from the neutral position to the injection position can be effected by moving the tip 222, 232 away from the fixed part and by bringing the presses 224, 234 closer to the fixed part. The tip in the neutral position can define a wall parallel to the longitudinal axis.

In the case where the device comprises two incision components, each component 220, 230 is mounted to move relative to the body of the device 1, between a neutral position and an injection position. The passage from the neutral position to the injection position can be effected by moving the tips 222, 232 apart and by moving the presses 224, 234 closer together. The tips in the neutral position can define a wall parallel to the longitudinal axis.

In the case where the device comprises three or more incision components, these are distributed in angular sectors around the longitudinal axis A. They are located facing one another, defining the ejection channel along the tips and the reservoir channel further toward the inside of the device. The components are all movable between a neutral position and an injection position. The passage from the neutral position to the injection position can be effected by moving the different tips apart and by moving the presses closer together. In a variant, the device comprises a plurality of these incision components, only a part of which is mounted so as to be movable relative to the device. This part of the components is movable between a neutral position and an injection position.

Different embodiments of components mounted between a neutral position and an injection position are possible.

### Pivot Zone

For example, a component 220, 230 can be mounted so as to be able to rotate relative to the body 150 of the device by means of a pivot zone 264, 274 which connects each incision component 220, 230 to the body 150.

The pivot zone allows a rotation of the component 220, 230 mounted so as to move in rotation with regard to the body 150 in the transverse direction of the axis C. The pivot zone is located outside a separation plane passing through the ejection channel. In particular, this separation plane can be defined by the longitudinal axis A and by the transverse axis C.

The pivot zone 264, 274 is located with regard to the separation plane between the ejection channel 280 and the reservoir channel 282. In particular, in the longitudinal direction, the pivot zone 264, 274 is located between the ejection channel 280 and the reservoir channel.

Thus positioned, the pivot zones allow a rotational movement of the components with regard to the body 150. This allows the tips to move apart or toward each other and the presses 224, 234 to move apart or toward each other. More precisely, when the tips move apart, the presses move toward each other and vice versa.

In the case where the device comprises three (or more) incision components, these are distributed in angular sectors around the longitudinal axis A. Each component is centered around a component plane which passes through the longitudinal axis. The planes are evenly distributed angularly around the longitudinal axis. Each component can then be mounted so that it can rotate relative to the body 150 in three (or more) directions which lie in the same plane of rotation perpendicular to the longitudinal axis A, each direction being orthogonal to the component plane associated with the component.

The pivot zone can be a pinched-in zone of material, i.e., a zone that has a sufficiently thin thickness of a material that is sufficiently flexible to allow elastic rotation around the transverse direction. A pivot zone made in the form of a pinched-in zone also allows defining the neutral position as a default position to which the components will tend to return in the absence of constraints.

In a variant, the pivot zone can be produced by any pivot connection along the transverse axis between the incision component and the body of the device. The device 1 can comprise a return component so that the components return to the neutral position by default, i.e., in the absence of constraints.

With reference to FIG. 2, the body 150 of the device can comprise two lateral wings 160, 170 which extend longitudinally toward the tips. The lateral wings are separated and facing each other in relation to the separation plane defined by the longitudinal axis A and the lateral axis B. The wings 160, 170 comprise at their longitudinal end on the side of the tips. Each tongue 162, 172 is connected to an incision component 220, 230 by a pivot zone 264, 274.

The tongues 162, 172 are laterally narrower than the wings 160, 170. The tongues extend longitudinally opposite the presses 224, 234. Each tongue is separated laterally from a press by a gap in material 164, 174.

The gaps 164, 174 communicate with a housing 180 defined by an inner contour of the wings 160, 170, the presses being located inside the housing 180. Since the presses are laterally separated to form the reservoir channel 282, the reservoir channel is in contact with the housing 180.

The device 1 is configured to move the components 220, 230 when the tips are applied to the skin of a human or animal body, from the neutral position to the injection position so that a volume of the ejection channel 280 is increased and a volume of the reservoir channel 282 is decreased.

In the case where the device comprises three (or more) incision components, the body 150 of the device can comprise three (or more) wings which are distributed along the same angular sectors as the sectors of the incision components so as to position an incision component opposite a wing.

Various embodiments of this movement when the tips are applied to the skin are possible.

### Levers

When each component 220, 230 is mounted so as to be rotatable relative to the body 150 of the device by means of a pivot zone 264, 274 which connects each incision component 220, 230 to the body 150, the device can, for example, comprise levers 260, 270 configured to move the components 220, 230 when the levers 260, 270 come into contact with the skin.

The levers 260, 270 are located on either side of the separation plane passing through the ejection channel.

Each lever 260, 270 is rigidly connected to a component 220, 230 in a rigid zone 266, 276. The rigid zone is especially more rigid than the pivot zone 264, 274 under the exertion of a torque along the transverse axis.

Each lever 260, 270 is also connected to the body 150 of the device 1 by one of the pivot zones 264, 274. In this case, each component is indirectly connected to the body 150 of the device via a rigid zone 266, 276, a lever 260, 270 and a pivot zone 264, 274.

The incision assembly 200 comprises levers 260, 270 in addition to the incision components 220, 230.

Each lever extends from the rigid zone in the lateral direction B. The rigid zone can lie outside the separation plane and in the longitudinal direction opposite an upper part of the ejection channel close to the reservoir channel.

Each lever 260, 270 extends from the rigid zone 266, 276 to one end 263, 273.

Each lever has an essentially flat outer surface 262, 272. In the neutral position of the components, this flat surface can be parallel to the lateral axis B and to the transverse axis C.

### Means of Locking the Lever

Optionally, the device comprises means for locking the levers 260, 270. In particular, these locking means can fix the levers in the injection position, once the volume of the ejection channel is increased and the volume of the reservoir channel is decreased.

To this end, and as can be seen in FIGS. 1 and 2, the incision device 1 comprises arms 265, 275 extending from ends 263, 273 of levers 260, 270. These arms extend in the longitudinal direction away from the tips 222, 232. These arms are located laterally opposite the wings 160 and 170 of the body 150 of the device 1. The longitudinal ends of the arms 265, 275 comprise hooks or lugs 267, 277 directed laterally toward the inside of the device 1, i.e., toward the longitudinal axis A.

Laterally opposite the hooks 267, 277 are located complementary hooks 293, 295 comprised on the body 150 of the device 1. The complementary hooks can, for example, be associated with material recesses 294, 296 in the lateral flanks of the wings 160, 170, which recesses have a shape complementary to the hooks 267, 277. These recesses produce, in correspondence with the hollows of the hooks 267, 277, projecting parts which are the complementary hooks 293, 295.

In the neutral position, the hooks 265, 275 are laterally outside the complementary hooks 293, 295.

### Component Walls

The component 220, 230 has a first wall 225, 235 which defines the reservoir channel 282. This first wall is located opposite a second wall of the fixed part of the device located opposite the component.

The first wall can have different shapes.

Preferably, the first wall 225, 235 has a shape complementary to the second wall of the device located opposite the first wall.

In the case where the device comprises two incision components and with reference to FIGS. 1 to 4, the components 220, 230 have walls 225, 235 opposite each other, these walls defining between them the reservoir channel 282 and having complementary shapes. The walls 225, 235 can, in particular, be walls of the presses 224, 234 mentioned above.

In a first option of this first variant, the walls 225, 235 opposite each other are straight. They are then preferably parallel to the longitudinal axis A or almost parallel to this axis. In a second option of this first variant, one of the two walls has a convex zone 228, 238 opposite a concave zone 226, 236 of the other of the two walls.

A convex zone of a first wall is understood here as a zone rounded toward the outside, i.e., toward the second wall.

A concave zone of a first wall is understood here as a zone rounded toward the inside, i.e., recessed toward the inside of the first wall.

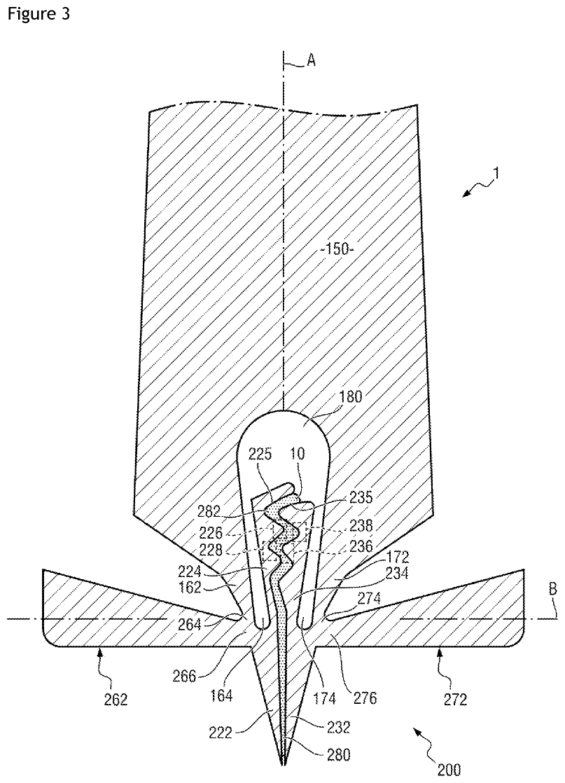

With reference to FIG. 3, the zone 226 of the wall 225 of the press 224 is a convex zone curved toward the outside of the press 224. The zone 226 is opposite the zone 238 of the wall 235 of the press 234. The zone 238 is a concave zone curved toward the inside of the press 234.

Still with reference to FIG. 3, the zone 228 of the wall 225 of the press 224 is a concave zone curved toward the inside of the press 224. The zone 228 is opposite the zone 236 of the wall 235 of the press 234. The zone 236 is a convex zone curved toward the outside of the press 234.

More precisely, the concavity and convexity mentioned can be limited to curvatures in the plane defined by the longitudinal axis A and the lateral axis B. In other words, the walls 225, 235 have no curvature in the transverse direction which is perpendicular to the longitudinal direction (i.e., the direction of extension of the tips) and to the lateral direction (i.e. the direction in which the tips are separated from each other). By limiting the curvature of the walls 225, 235 in the plane defined by the longitudinal axis A and the lateral axis B, the incision components are easier to manufacture.

One of the two walls can have a convex zone and a concave zone respectively opposite a concave zone and a convex zone of the other of the two walls. The walls have corrugations around a main direction, which can be the longitudinal direction. The walls are zigzag-shaped in the plane defined by the longitudinal axis A and the lateral axis B.

It should also be noted that the part of the reservoir channel 282 toward the inside of the device has a very flared outlet. In other words, in the part of the reservoir channel 282 as one moves away from the tips along the longitudinal axis, the walls defining the reservoir channel move away rapidly from each other. This highly flared outlet defines a limit on the liquid level. It would be very expensive in terms of surface energy for the liquid to be located in this flared outlet. The more the liquid moves into this flared outlet, the greater the energy cost.

### Superficial Incision Process

A device for injecting into the skin by superficial incision as has just been presented makes it possible to implement a superficial incision method according to the invention.

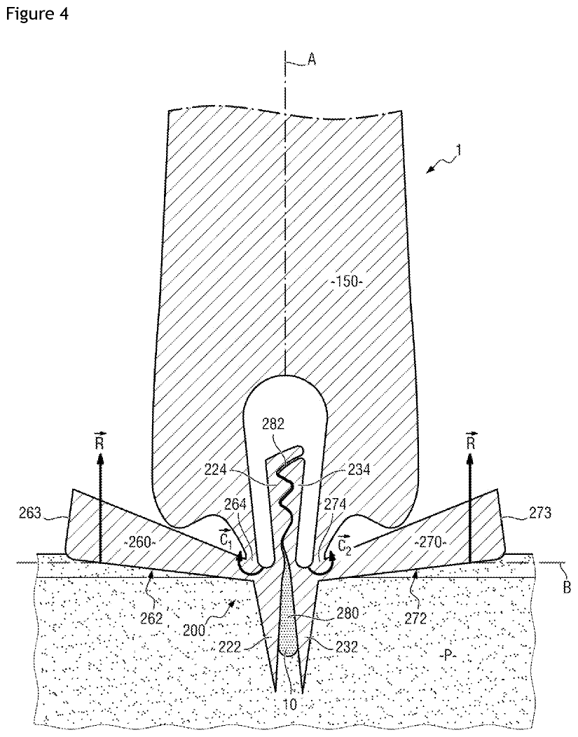

With reference to FIGS. 3 and 4, a mode of implementation of this method will be presented.

In an optional preliminary step of loading the device, the tips of the device can be soaked in an active liquid (vaccine solution or allergological test liquid). In this way, a quantity of liquid is drawn into the ejection channel and into the reservoir channel by capillary action.

It should be noted that in the case where the walls defining the reservoir channel have a convex zone 228, 238 opposite a concave zone of the other of the two walls, this allows the reservoir channel to have a small lateral width so that more liquid 10 can be drawn into the reservoir channel.

Furthermore, the thickness of the incision components and especially of the presses in the transverse direction of the transverse axis C is adjustable. This is chosen according to the volume of liquid that it is desired to be drawn into the reservoir channel when the device is loaded.

Capillary suction of the active liquid into the ejection channel and into the reservoir channel assumes a sufficiently small lateral gap between the tips and the presses. In this respect and with reference to FIG. 1, the device has a slot 104 which separates the device into two halves 108 and 110. The handle 100, when it is present, can especially have the shape of a clover split in its innermost part of the device 1 along the longitudinal axis A. The slot 104 can pass through the entire device 1 from the clover. The presence of the slot and the clover especially makes it possible, during manufacture by molding, to maximize the angular narrowing of the two halves 108, 110 when the component is cooled at the end of the manufacturing process. This reduces the lateral gap between the tips and the presses. It is possible for this gap to assume a predetermined nominal value by using stops 112 to set a limit to the angular narrowing.

It should be noted that the stops 106 make it possible to hold the halves 108 and 110 opposite each other without possible transverse deviation in the direction of the transverse axis C.

According to the method, the tips 222, 232 of a device 1 as described above are applied to the skin P of a human or animal body.

The situation before the tips come into contact with the skin is illustrated in FIG. 3. The components 220, 230 are in neutral position. In this position the inner walls of the tips are parallel to the longitudinal axis.

Before the tips come into contact with the skin, the levers 260, 270 define outer surfaces 262, 272 perpendicular to the longitudinal axis.

The situation after the tips have come into contact with the skin is illustrated in FIG. 4.

The tips come into contact with the skin P and penetrate into the epidermis. The outer surfaces 262, 272 of the levers 260, 270 come into contact with the surface of the skin.

The application of the device to the skin causes a rotational torque which is applied to the incision assembly 200. While the outer surfaces 262, 272 are retained outside the skin according to a return force R which is directed toward the outside of the skin, the tips are sharp enough not to be retained by the skin. The push back of the skin on the incision assembly does not apply uniformly in a plane perpendicular to the longitudinal axis. This tends to rotate each incision component and lever pair around the pivot zone associated with it according to pairs C1 and C2 shown in FIG. 4.

The ends 263, 273 of the levers 260, 270 are raised longitudinally toward the inside of the device and the tips penetrate further into the epidermis, i.e., by moving away from the device.

The incision components are rotated by the levers to the incision position. The tips 222, 232 move laterally outwards so that the ejection channel 280 enlarges. Simultaneously, the presses 224, 234 come closer together and come into contact with each other so that the reservoir channel 282 decreases. The injection position is attained.

In this regard, the use of complementary shapes of the walls defining the reservoir channel makes it possible to further reduce the volume of the reservoir channel during the passage from the neutral position to the injection position.

The liquid 10 initially contained in the reservoir channel leaves the reservoir channel, whose volume decreases. The liquid does not escape through the flared part. The liquid is expelled toward the ejection channel 280.

In this way, a greater quantity of active product is ensured which penetrates the skin and the situation where liquid is retained inside the device is avoided.

It should be noted that the surfaces 262, 272 guarantee both that the tips 220 and 230 are fully inserted into the skin during application and that they do not penetrate too deeply into the tissues of the human or animal body.

If the device comprises means for locking the levers 260, 270, then during the movement of the levers 260, 270 from the neutral position to the injection position, the hooks 267, 277 cause the hooks 293, 295 to move inwards by elastic deformation. Once the hooks 267, 277 have passed the hooks 293, 295, the hooks 293, 295 return elastically to their initial position and hold the hooks 267, 277 and consequently the levers 260, 270 in the injection position. This makes it possible to keep the tips 222, 232 spaced apart when the device 1 is then withdrawn and to prevent active liquid from being drawn back into the ejection channel 280.

1. A device for injecting into the skin by superficial incision, the device comprising
an incision component comprising a tip oriented toward the outside of the device,
the tip defining an ejection channel,
the incision component defining a reservoir channel,
the device being configured to move the component when the tip is applied to the skin of a human or animal body, such that a volume of the ejection channel is increased and a volume of the reservoir channel is decreased.

2. The device according to claim 1, wherein the incision component is movably mounted relative to a body of the device, the incision component comprising a press located between the tip and the body of the device, the press defining the reservoir channel.

3. The device according to claim 2, wherein the tip is located on a first side of a separation plane passing through the ejection channel, the incision component being mounted so as to be rotatable relative to the body of the device along a pivot, the pivot zone being located outside the separation plane between the ejection channel and the reservoir channel.

4. The device according to claim 3, wherein the press is located inside a housing defined by wings of the body.

5. The device according to claim 3, comprising a lever, the lever being located on the first side of the separation plane, the lever being rigidly connected to the component and the lever being connected to the body by the pivot zone, the lever being configured to move the component when the lever comes into contact with the skin.

6. The device according to claim 2, comprising means for locking the lever.

7. The device according to claim 6, wherein the locking means comprises complementary hooks on the body of the device and on an arm extending from one end of the lever.

8. The device according to claim 1, wherein the component comprises a first wall, the first wall defining the reservoir channel, the first wall having a shape complementary to a second wall of the device located opposite the first wall.

9. The device according to claim 8, wherein one of the first wall and the second wall has a convex zone opposite a concave zone of the other wall.

10. A method of surface incision wherein a tip of a device for injecting into the skin by surface incision is applied to the skin of a human or animal body, the application causing a reduction in a volume of a reservoir channel and an increase in a volume of an ejection channel, the ejection channel being defined by the tip, so as to transport a liquid from the reservoir channel into the ejection channel.
